(19) United States
(12) Patent Application Publication
(10) Pub. No.: US 2024/0055113 A1
(43) Pub. Date: Feb. 15, 2024

(54) DEVICE CONTROL FOR BIOLOGICAL SAMPLE ANALYSIS

(71) Applicant: Radiometer Medical ApS, Brønshøj (DK)

(72) Inventors: Jacob Ranselaar HANSEN, Brønshøj (DK); Morten HASTRUP, Brønshøj (DK)

(21) Appl. No.: 18/268,355

(22) PCT Filed: Dec. 20, 2021
(86) PCT No.: PCT/EP2021/086838

§ 371 Date: Jun. 20, 2023

(30) Foreign Application Priority Data
Dec. 21, 2020 (EP) 20216074.3

(51) Int. Cl. G16H 40/40

(52) U.S. Cl. CPC G16H 40/40

(57) ABSTRACT

A computer-implemented method of controlling operator interaction with one or more operator devices is disclosed. The one or more operator devices comprise one or more analyzing devices configured to analyze biological samples. The method comprises, responsive to any of the one or more analyzing devices being triggered by the operator to perform an analysis of a sample, acquiring—in association with an identifier of the operator—information regarding any sample associated handling error detected by the triggered analyzing device. The method also comprises dynamically updating handling error data associated with the identifier of the operator based on the information regarding detected handling errors, and controlling—for the identifier of the operator—interaction with at least one of the one or more operator devices based on the handling error data associated with the identifier of the operator. A computer-implemented method of an analyzing device is also disclosed. The method comprises, responsive to the analyzing device being triggered by the operator to perform an analysis of a sample, detecting any sample associated handling error. The method also comprises, in response to detection of a handling error, providing—in association with an identifier of the operator—information regarding the detected handling error for dynamic updating of handling error data associated with the identifier of the operator. Corresponding apparatus, server, storage device, analyzing device, operator device, system, and computer program product are also disclosed.

### TECHNICAL FIELD

The present disclosure relates generally to the field of devices configured to analyze biological samples. More particularly, it relates to control of operator interaction with devices in the context of biological sample analysis.

### BACKGROUND

Within the field of clinical analysis, a wide variety of electronic devices are known for the acquisition and registration of patient-related data. U.S. Pat. No. 8,608,654 B2 describes some general aspects of an example system for acquiring patient-related data.

One example of electronic devices for acquisition and registration of patient-related data relates to analyzing devices configured to analyze biological samples. Such analyzing devices may, for example, be deployed in a laboratory environment or in a point of care (POC) environment.

Typically, an analyzing device may comprise a sample input for receiving a biological sample, and a sample processing arrangement for performing analysis of the biological sample. Furthermore, an analyzing device may comprise an operator interface (e.g., a rendering and/or operator input device, such as a touch screen), and/or a result output for providing a result of the analysis of the sample.

Various types of analyzing devices are well known in the art and their general structure and function will not be elaborated on or exemplified further herein. For example, WO 2015/071419 A1 describes operator-specific adaptation of a medical analyzer user interface.

One or more sample handling errors performed by the operator before, or in association with, inputting the biological sample to the analyzing device may result in inferior analysis (e.g., one or more of: reduced accuracy of the analysis result, invalid analysis result, and lack of analysis result—for example, due to interrupted sample processing). Thus, controlling (preferably minimizing, or at least reducing) the occurrence of sample handling errors is desirable.

Such control may be particularly difficult in a POC environment where the circumstances for proper sample handling may be inferior (e.g., in terms of parameters such as temperature, lighting, sanitation, etc.), and/or where numerous different operators (possibly with varying experience and/or professional role) may have access to the analyzing device.

Therefore, there is a need for new approaches that enable control of the occurrence of sample handling errors.

### SUMMARY

It should be emphasized that the term “comprises/comprising” (replaceable by “includes/including”) when used in this specification is taken to specify the presence of stated features, integers, steps, or components, but does not preclude the presence or addition of one or more other features, integers, steps, components, or groups thereof. As used herein, the singular forms “a”, “an” and “the” are intended to include the plural forms as well, unless the context clearly indicates otherwise.

Generally, when an arrangement is referred to herein, it is to be understood as a physical product; e.g., an apparatus. The physical product may comprise one or more parts, such as controlling circuitry in the form of one or more controllers, one or more processors, or the like.

It is an object of some embodiments to solve or mitigate, alleviate, or eliminate at least some of the above or other disadvantages.

A first aspect is a computer-implemented method of controlling operator interaction with one or more operator devices, wherein the one or more operator devices comprise one or more analyzing devices configured to analyze biological samples.

The method comprises, responsive to any of the one or more analyzing devices being triggered by the operator to perform an analysis of a sample, acquiring—in association with an identifier of the operator—information regarding any sample associated handling error detected by the triggered analyzing device.

The method also comprises dynamically updating handling error data associated with the identifier of the operator based on the information regarding detected handling errors, and controlling (for the identifier of the operator) interaction with at least one of the one or more operator devices based on the handling error data associated with the identifier of the operator.

In some embodiments, the information regarding a handling error comprises indication of one or more of: detection of the handling error, and an error type of the handling error.

In some embodiments, the identifier of the operator comprises one or more of: an individual identifier of the operator, and a group identifier of the operator.

In some embodiments, the identifier of the operator is detected based on one or more of: an account accessed by the operator when using the analyzing device, and an application module accessed by the operator when using the analyzing device.

In some embodiments, the method further comprises acquiring—in association with the identifier of the operator and the information regarding a handling error—one or more of: identification of the analyzing device triggered by the operator to perform the analysis, identification of an equipment type of the analyzing device triggered by the operator to perform the analysis, and identification of an analysis type of the analysis performed.

In some embodiments, dynamically updating the handling error data associated with the identifier of the operator is further based on one or more of: the identification of the analyzing device triggered by the operator to perform the analysis, the identification of the equipment type of the analyzing device triggered by the operator to perform the analysis, and the identification of the analysis type of the analysis performed.

In some embodiments, the handling error data comprises a number, or a ratio, or a percentage, of handling errors for the identifier of the operator.

In some embodiments, controlling interaction with at least one of the one or more operator devices comprises one or more of: prohibiting or restricting further access to the one or more analyzing devices, for the identifier of the operator, prohibiting performance of the analysis for further samples, for the identifier of the operator, increasing an amount of user interface rendered guidance instructions related to the one or more analyzing devices and/or relating to the analysis, for the identifier of the operator, and enforcing or prompting training associated with the analyzing device and/or associated with the analysis, for the identifier of the operator.

In some embodiments, controlling interaction with at least one of the one or more operator devices comprises: determining a score value for the identifier of the operator based on the dynamically updated handling error data, and controlling interaction with at least one of the one or more operator devices based on the score value.

In some embodiments, the method further comprises one or more of: rendering a notification based on the score value for one or more operator identifiers, and tracking the score value over time for one or more operator identifiers.

A second aspect is a computer-implemented method of an analyzing device configured to analyze biological samples, the method being for controlling operator interaction with one or more operator devices, wherein the one or more operator devices comprise the analyzing device.

The method comprises, responsive to the analyzing device being triggered by the operator to perform an analysis of a sample, detecting any sample associated handling error.

The method also comprises, in response to detection of a handling error, providing—in association with an identifier of the operator—information regarding the detected handling error for dynamic updating of handling error data associated with the identifier of the operator. The handling error data associated with the identifier of the operator is for controlling interaction with at least one of the one or more operator devices, for the identifier of the operator.

In some embodiments, controlling interaction with at least one of the one or more operator devices comprises one or more of: prohibiting or restricting further access to the one or more analyzing devices, for the identifier of the operator, prohibiting performance of the analysis for further samples, for the identifier of the operator, increasing an amount of user interface rendered guidance instructions related to the one or more analyzing devices and/or relating to the analysis, for the identifier of the operator, and enforcing or prompting training associated with the analyzing device and/or associated with the analysis, for the identifier of the operator.

In some embodiments, the method further comprises, responsive to the analyzing device being triggered by the operator to perform the analysis of the sample, acquiring a control instruction associated with the identifier of the operator, and controlling interaction with the analyzing device by the operator based on the control instruction. The control instruction is based on handling error data as updated based on information regarding previously detected handling errors associated with the identifier of the operator.

A third aspect is a computer program product comprising a non-transitory computer readable medium, having thereon a computer program comprising program instructions. The computer program is loadable into a data processing unit and configured to cause execution of the method according to any of the first and second aspects when the computer program is run by the data processing unit.

A fourth aspect is an apparatus for controlling operator interaction with one or more operator devices, wherein the one or more operator devices comprise one or more analyzing devices configured to analyze biological samples.

The apparatus comprises controlling circuitry configured to cause acquisition, in response to any of the one or more analyzing devices being triggered by the operator to perform an analysis of a sample, of information—in association with an identifier of the operator—regarding any sample associated handling error detected by the triggered analyzing device.

The controlling circuitry is also configured to cause dynamic update of handling error data associated with the identifier of the operator based on the information regarding detected handling errors, and control (for the identifier of the operator) of interaction with at least one of the one or more operator devices based on the handling error data associated with the identifier of the operator.

In some embodiments, control of interaction with at least one of the one or more operator devices comprises one or more of: prohibition or restriction of further access to the one or more analyzing devices, for the identifier of the operator, prohibition of performance of the analysis for further samples, for the identifier of the operator, increase of an amount of user interface rendered guidance instructions related to the one or more analyzing devices and/or relating to the analysis, for the identifier of the operator, and enforcement or prompt of training associated with the analyzing device and/or associated with the analysis, for the identifier of the operator.

A fifth aspect is a server comprising the apparatus of the fourth aspect.

A sixth aspect is a storage device carrying handling error data for controlling operator interaction with one or more operator devices according to any of the first, second, third, and fourth aspects, wherein the one or more operator devices comprise one or more analyzing devices configured to analyze biological samples. The handling error data is associated with respective identifiers of a plurality of operators, and is based on information regarding sample associated handling errors detected responsive to any of the one or more analyzing devices being triggered by an operator to perform an analysis of a sample. A seventh aspect is an analyzing device configured to analyze biological samples. The analyzing device comprises controlling circuitry configured to cause detection, in response to the analyzing device being triggered by an operator to perform an analysis of a sample, of any sample associated handling error. The controlling circuitry is also configured to cause provision, in response to detection of a handling error, of information—in association with an identifier of the operator—regarding the detected handling error for dynamic updating of handling error data associated with the identifier of the operator. The handling error data associated with the identifier of the operator is for controlling interaction with at least one of one or more operator devices, for the identifier of the operator, wherein the one or more operator devices comprise the analyzing device.

In some embodiments, controlling interaction with at least one of the one or more operator devices comprises one or more of: prohibiting or restricting further access to the one or more analyzing devices, for the identifier of the operator, prohibiting performance of the analysis for further samples, for the identifier of the operator, increasing an amount of user interface rendered guidance instructions related to the one or more analyzing devices and/or relating to the analysis, for the identifier of the operator, and enforcing or prompting training associated with the analyzing device and/or associated with the analysis, for the identifier of the operator.

An eighth aspect is an operator device, wherein the operator device is an analyzing device configured to analyze biological samples and/or a training device configured to enable sample handling training for biological sample analysis. The operator device comprises controlling circuitry configured to cause acquisition of a control instruction associated with an identifier of an operator, and control of interaction with the operating device by the operator based on the control instruction. The control instruction is based on handling error data as updated based on information regarding previously detected handling errors associated with the identifier of the operator.

In some embodiments, control of interaction with at least one of the one or more operator devices comprises one or more of: prohibition or restriction of further access to the one or more analyzing devices, for the identifier of the operator, prohibition of performance of the analysis for further samples, for the identifier of the operator, increase of an amount of user interface rendered guidance instructions related to the one or more analyzing devices and/or relating to the analysis, for the identifier of the operator, and enforcement or prompt of training associated with the analyzing device and/or associated with the analysis, for the identifier of the operator.

A ninth aspect is a system for controlling operator interaction with one or more operator devices, wherein the one or more operator devices comprise one or more analyzing devices configured to analyze biological samples. The system comprises the server of the fifth aspect, the storage device of the sixth aspect, and at least one analyzing device according to the seventh aspect.

In some embodiments, any of the above aspects may additionally have features identical with or corresponding to any of the various features as explained above for any of the other aspects.

An advantage of some embodiments is that approaches are provided that enable control (and preferably reduction) of the occurrence of sample handling errors.

An advantage of some embodiments is that quality control of sample handling is provided.

An advantage of some embodiments is that control of interaction with at least one of the one or more operator devices which may comprise prohibition or restriction of further access to the one or more analyzing devices may ensure regulatory compliance.

An advantage of some embodiments is that the prohibition or restriction of further access to the one or more analyzing devices may ensure that only trained operators and/or operators associated with an acceptable level of sample handling errors compared to a population of operators are allowed access to the one or more analyzing devices which in turn may reduce the occurrence of sample handling errors and may provide improved quality control of sample handling.

An advantage of some embodiments is that the provided approaches that enable control (and preferably reduction) of the occurrence of sample handling errors enable a data driven continuous learning of the level of training among a population of operators.

An advantage of some embodiments is that since the continuous learning is population based and data driven a more personalized, specific, and efficient training may be provided to an operator.

An advantage of some embodiments is that since the continuous learning is population based and data driven it may be advantageously implemented with machine learning.

### BRIEF DESCRIPTION OF THE DRAWINGS

Further objects, features and advantages will appear from the following detailed description of embodiments, with reference being made to the accompanying drawings. The drawings are not necessarily to scale, emphasis instead being placed upon illustrating the example embodiments.

FIG. 1 is a signaling diagram combined with a collection of flowcharts illustrating example method steps and signaling according to some embodiments;

FIG. 2 is a schematic drawing illustrating example mechanisms according to some embodiments;

FIG. 3 is a schematic block diagram illustrating example functional modules according to some embodiments;

FIG. 4 is a schematic block diagram illustrating an example system according to some embodiments;

FIG. 5 is a schematic block diagram illustrating an example apparatus according to some embodiments;

FIG. 6 is a schematic block diagram illustrating an example operator device according to some embodiments; and

FIG. 7 is a schematic drawing illustrating an example computer readable medium according to some embodiments.

### DETAILED DESCRIPTION

As already mentioned above, it should be emphasized that the term “comprises/comprising” (replaceable by “includes/including”) when used in this specification is taken to specify the presence of stated features, integers, steps, or components, but does not preclude the presence or addition of one or more other features, integers, steps, components, or groups thereof. As used herein, the singular forms “a”, “an” and “the” are intended to include the plural forms as well, unless the context clearly indicates otherwise.

Embodiments of the present disclosure will be described and exemplified more fully hereinafter with reference to the accompanying drawings. The solutions disclosed herein can, however, be realized in many different forms and should not be construed as being limited to the embodiments set forth herein.

In the following, embodiments will be described for control (e.g., reduction) of the occurrence of sample handling errors in the context of using analyzing devices configured to analyze biological samples. According to some embodiments, this is achieved by controlling operator interaction with one or more operator devices.

Generally, an operator device may be an analyzing device (e.g., a point of care, POC, device) and/or a training device. Training may be performed on an analyzing device, on a simulation/demo device, or on a general purpose device (such as a smartphone or computer, for example). Thus, the training device may be an analyzing device, a simulation/demo device, or a general purpose device. A training device may be configured to enable sample handling training for biological sample analysis.

Generally, when a biological sample is referred to herein, it is meant to encompass any suitable biological sample. Example biological samples include blood samples, saliva samples, urine samples, biopsy samples, etc.

Also generally, when a sample handling error is referred to herein, it is meant to encompass any suitable sample handling error. Typically, a sample handling error is an error which is likely to have been caused by the operator (e.g., a pre-analytical error). For example, a sample handling error may be caused by an operator by mistake, due to lack of experience, due to lack of training, or on purpose. A sample handling error may be caused by the operator before, or in association with, inputting the biological sample to the analyzing device.

Some example indications of pre-analytical errors and possible therefore are tabulated below.

| Indication of pre-analytical error | Possible sources |
|---|---|
| Test request | Erroneous request, inappropriate request |
| Patient identification | Wrong identification, missing identification |
| Patient preparation and condition | Biological variables, diet, fasting |
| Time of specimen collection | Diurinal, fasting, peak and trough |
| Specimen collection and | Mislabeled, unlabeled, hemolysis, incorrect order of |
| identification | draw, wrong tube type, wrong anticoagulant, IV line |
| | (contamination from infusion), tourniquet time, |
| | posture, fist clenching, traumatic draw, insufficient or |
| | vigorous tube mixing, quantity not sufficient, clot |
| Sample transportation | Length and environmental conditions, excessive |
| | transportation time, temperature, pneumatic tube |
| | system |
| Sample storage | Improper storage, length of time, temperature, |
| | evaporation, freezing and thawing |
| Sample preparation | Delayed processing, aliquoting, centrifugation |
| | conditions |

Generally, a sample handling error may be (explicitly or implicitly) detected by the analyzing device.

A sample handling error including faulty execution when inputting the sample to the analyzing device may, for example, be detected by a sample inlet being erroneously operated (e.g., not being closed) and/or by the sample being erroneously inserted (e.g., improper orientation of a sample holder, or sample missing).

A sample handling error including improper management (e.g., keeping it at wrong temperature, shaking it too much or too little, extracting a too small amount of it from the patient, or letting too long time pass between extraction from patient to insertion to analyzing device) of the sample before inputting the sample to the analyzing device may, for example, be detected by one or more of: sample size being out-of-range, sample temperature being out-of-range, bubbles and/or clots being present in the sample, and sample time stamp being out-of-range. The term out-of-range generally refers to a parameter value falling below or above a range of acceptable values for the parameter.

Generally, when an operator is referred to herein, it is meant to encompass any suitable person interacting with the analyzing device. Example operators include: physicians, nurses, assisting nurses, care-giving assistants, laboratory technicians, and laboratory assistants. In fact, even patients and their relatives (or other non-medically trained personnel) may be considered as operators when the analyzing device is configured for self-care.

An operator is associated with at least one operator identifier (i.e., an identifier of the operator). The identifier of the operator may be an individual identifier, and/or a group identifier. An individual identifier may, for example, comprise an operator identity (e.g., defined via one or more of: a user account, a user application instantiation, a user identification number, a user radio frequency identification— RFID, or similar). A group identifier may, for example, comprise an identity of a group that the operator belongs to (e.g., a hospital, a laboratory, a department, a profession—physician/nurse/etc., an experience level—inexperienced/experienced/expert, time span served in profession, time span served at the hospital/laboratory, time span using the type of analyzing device, frequency of using the type of analyzing device, etc.). A group identifier may be seen as an identifier of an operator type.

Also generally, when operator interaction is referred to herein, it is meant to encompass any suitable interaction by the operator with the operator device. Example operator interaction includes the operator using the analyzing device to perform an analysis of a sample, training on the analyzing device (or on another training device) to perform an analysis of a training sample, conducting an interactive training session via a general purpose device, completing a questionnaire enabled via a general purpose device, and taking part of instructional content (e.g., watching an instruction video) enabled via a general purpose device.

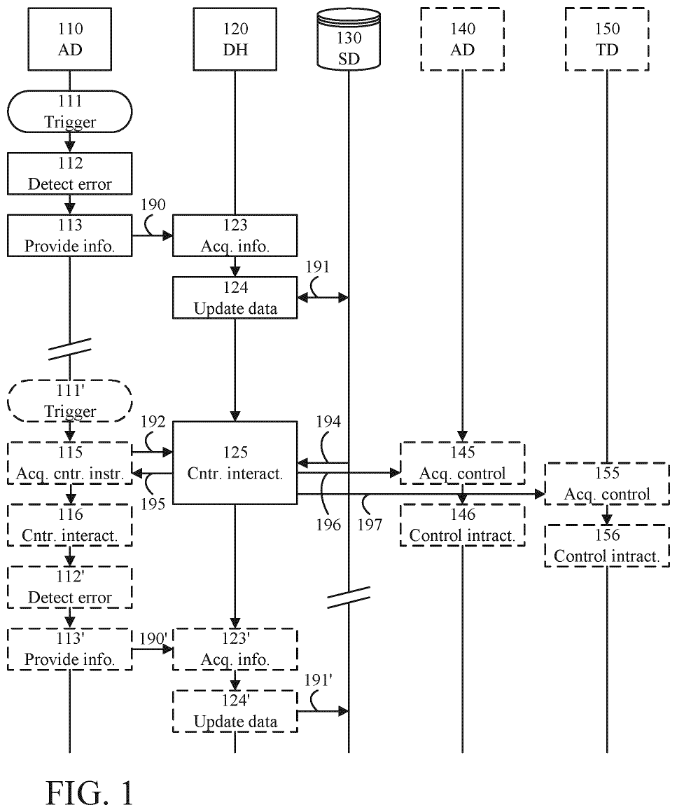

FIG. 1 illustrates example computer-implemented methods and signaling according to some embodiments. The methods and signaling of FIG. 1 are described in a context involving a first analyzing device (AD) 110, a data handler (DH) 120, and a storing device (SD) 130. Optionally, the context may also involve a second analyzing device (AD) 140, and/or a training device (TD) 150.

The first and second analyzing devices 110, 140 and the training device 150 are examples of operator devices. Each of the first and second analyzing devices 110, 140 are configured to analyze biological samples.

Each of the data handler 120 and/or the storing device 130 may be comprised in a (same or different) server and/or may be comprised in a cloud-based deployment. For example, the data handler 120 and/or the storing device 130 may be comprised in a central device of a hospital information system (HIS) or a laboratory information system (LIS).

The example computer-implemented methods and signaling exemplified in FIG. 1 are for controlling operator interaction with one or more of the operator devices 110, 140, 150. One purpose of such control may be to control (e.g., reduce) the occurrence of sample handling errors in the context of using the analyzing devices 110, 140.

A computer-implemented method of the first analyzing device 110 starts responsive to the first analyzing device 110 being triggered by an operator to perform an analysis of a sample, as illustrated by 111. For example, the method of the first analyzing device 110 may commence by detecting that the first analyzing device 110 is triggered to perform an analysis of a sample.

The triggering may be defined and/or detected in any suitable way. Example triggering includes switching on the first analyzing device 110, waking up the first analyzing device 110 from a low power mode, causing the first analyzing device 110 to enter an operational mode, initiating analysis of a sample (e.g., by entering an input relating to the analysis via a user interface of the first analyzing device 110 and/or by inserting the sample into a sample inlet of the first analyzing device 110).

In step 112, any sample associated handling errors are detected by the first analyzing device 110. The sample associated handling errors may be any suitable sample associated handling errors, as has been exemplified above. The detection may be any suitable detection (e.g., related to any error message and/or error code provided by the analyzing device). Various possible specifics of the detection are well known and will not be elaborated on further herein.

Step 112 may be performed at any suitable time following the triggering 111 and relating to analysis of the sample. For example, step 112 may be performed directly responsive to the triggering 111, and/or before the analysis of the sample starts, and/or during the analysis of the sample, and/or responsive to the analysis of the sample being aborted, and/or responsive to the analysis of the sample being completed. Thus, the analysis of the sample may take place before, and/or during, and/or after execution of step 112.

In response to detection of a handling error in step 112, the first analyzing device 110 provides information regarding the detected handling error in association with an identifier of the operator, as illustrated by step 113. The information regarding the detected handling error associated with an identifier of the operator is illustrated as a signal 190 sent from the first analyzing device 110 to the data handler 120.

As mentioned before, the identifier of the operator may be an individual identifier of the operator, and/or a group identifier of the operator. The identifier of the operator may be detected in connection with the triggering 111. For example, the identifier of the operator may be detected based on an account accessed by the operator when using the analyzing device (e.g., a user login), and an application module accessed by the operator when using the analyzing device (e.g., a profession associated application).

Step 113 may be performed at any suitable time following the detection 112. For example, step 113 may be performed directly responsive to the detection 112 (e.g., sending a signal 190 for each detected handling error), and/or responsive to the analysis of the sample being aborted (e.g., sending a signal 190 for each triggering 111; possibly associated with several handling errors), and/or responsive to the analysis of the sample being completed (e.g., sending a signal 190 for each triggering 111; possibly associated with several handling errors). In some embodiments, a single performance of step 113 is made for detected handling errors of more than one triggering (e.g., sending a signal 190 only for some of the triggers; possibly associated with several handling errors from different triggers).

The signal 190 is indicative of the identifier of the operator and the information regarding the detected handling error(s). The information regarding a detected handling error may comprise an indication that the handling error was detected (e.g., a count increment or a count value) and/or an error type of the handling error (e.g., an error code or similar identifier).

In one example, the signal 190 may comprise an operator identification and an error code (implicitly indicating that one instance of this error type was detected). In one example, the signal 190 may comprise an operator identification and an error flag (indicating that at least one error was detected; possibly without information of the type of error). In one example, the signal 190 may comprise an operator identification and an error count value (indicating the number of errors that was detected; possibly without information of the type of error). In one example, the signal 190 may comprise an operator identification, one or more error codes, and an error count value associated with each of the error codes (explicitly indicating the number of instances detected for each of the error types). Other ways to define the content of the signal 190 are also possible.

Each error type may correspond to a specific possible handling error, or may encompass several different possible handling errors. In the latter case, one example is when possible handling errors are grouped in terms of severity (e.g., causing less accurate analysis result, causing analysis interruption, and causing erroneous analysis result) and each error type corresponds to any error with a certain severity.

In some embodiments, the first analyzing device 110 provides further information to the data handler 120 (e.g., via the signal 190) in association with the identifier of the operator and the information regarding a handling error.

Such further information may, for example, comprise (explicit or implicit) identification of the first analyzing device 110, and/or (explicit or implicit) identification of an equipment type of the first analyzing device 110 (e.g., manufacturer, model, version, etc.),

In some embodiments, (explicit or implicit) identification of an analysis type of the triggered/performed analysis is provided (e.g., via the signal 190) by the first analyzing device 110 to the data handler 120. For example, the analysis type may be defined by the type of sample (e.g., blood, urine, etc.), and/or by the sought parameters (e.g., cholesterol value, egg white presence, etc.).

In some embodiments, a trigger count increment/value associated with the identifier of the operator is provided (e.g., via the signal 190) by the first analyzing device 110 to the data handler 120. Thus, the data handler 120 is informed of how many times the operator has triggered the first analyzing device; regardless of whether any handling error was detected.

A computer-implemented method of the data handler 120 also starts responsive to the first analyzing device 110 being triggered by an operator to perform an analysis of a sample, as illustrated by 111. The triggering may be detected by the data handler 120 in any suitable way. For example, the method of the data handler may commence by detecting the signal 190, and thereby implicitly detecting that the first analyzing device 110 has been triggered to perform an analysis of a sample.

In step 123, information regarding any sample associated handling error detected by the triggered analyzing device is acquired by the data handler 120, in association with an identifier of the operator. The information regarding the detected handling error associated with an identifier of the operator is illustrated as a signal 190 received by the data handler 120 from the first analyzing device 110. Typically, the data handler 120 acquires information regarding any sample associated handling error detected from a plurality of analyzing devices and/or in association with identifiers of a plurality of operators.

In step 124, the data handler 120 updates handling error data associated with the identifier of the operator based on the information regarding detected handling errors. In some embodiments, updating the handling error data in step 124 may be further based on an identification of the first analyzing device 110, and/or an identification of the equipment type of the first analyzing device 110, and/or the identification of the analysis type of the analysis triggered/performed.

An update may, for example, comprise determining an updated handling error data value based on a previous handling error data value and the information regarding detected handling errors. Thus, a handling error data value may be based on previously detected handling errors as well as currently detected handling errors.

A handling error data value may, for example, comprise an accumulated number of errors (e.g., in total or over a time window), an average number of errors per trigger (e.g., in total or over a time window), or a filtered average number of errors per trigger (e.g., phasing out errors as they get older).

The updating of step 124 is dynamic (i.e., the handling error data is varying over time based on acquisitions of new information regarding detected handling errors). For example, step 124 may be performed every time information regarding any sample associated handling error is acquired (e.g., every time a signal 190 is received), or more seldom (e.g., at regular time intervals). In FIG. 1, the update of handling error data is exemplified by interaction 191 between the data handler 120 and a storing device 130 holding the handling error data.

Step 124 may comprise performing calculations. For example, the data handler may read a current number (or ratio) value related to handling errors for the identifier of the operator from the storing device, calculate an updated number (or ratio) value related to handling errors for the identifier of the operator based on the acquired information, and replace the current number (or ratio) value with the updated number (or ratio) value for the identifier of the operator in the storing device.

It should be noted that, in some embodiments (e.g., when the signal 190 relates to several handling errors from different triggers), part of such calculations may be performed by the first analyzing device 110. For example, the first analyzing device 110 may calculate an average value of handling errors for the identifier of the operator per triggering of the first analyzing device 110. Thus, the information provided to the data handler may comprise a result of such partial calculations.

In step 125, the data handler 120 controls interaction (for the identifier of the operator) with at least one operator device 110, 140, 150 based on the handling error data associated with the identifier of the operator. In FIG. 1, 194 illustrates that the data handler 120 may extract the handling error data associated with the identifier of the operator as part of step 125.

In some embodiments, step 125 (and/or step 124) may comprise determining a score value for the identifier of the operator based on the dynamically updated handling error data, and using the score value for interaction control. Score values may, for example, be set in relation to threshold values for number/ratio of handling errors for the identifier of the operator (e.g., threshold values which have static values or a dynamic values, such as a percentile of the number/ratio of handling errors per operator in an operator population).

The score value may be individual to the operator or collective for a group of operators. Alternatively or additionally, the score value may be a collective score value for all handling error types, or may comprise a plurality of score values associated with respective handling error types. Alternatively or additionally, the score value may be a collective score value for all analyzing device types, or may comprise a plurality of score values associated with respective analyzing device types. Alternatively or additionally, the score value may be a collective score value for all analysis types, or may comprise a plurality of score values associated with respective analysis types.

Controlling interaction with an operator device which is an analyzing device may comprise prohibiting further access to the analyzing device for the identifier of the operator. Prohibition may, for example, be performed when a number, or ratio, of handling errors for the identifier of the operator is too high (e.g., higher than a threshold value—which may have a static value such as zero or another value, or a dynamic value such as a percentile of the number, or ratio, of handling errors per operator in an operator population). Alternatively or additionally, prohibition may be performed when a handling error score for the identifier of the operator is too low (e.g., lower than a threshold value—which may have a static value or a dynamic value such as a percentile of the handling error score per operator in an operator population). A previously enforced prohibition may be released when the number/ratio/score reaches an acceptable level again (e.g., defined by a threshold value, which may be the same as, or different from, the threshold value for prohibition).

Controlling interaction with an operator device which is an analyzing device may comprise restricting further access to the analyzing device for the identifier of the operator. Restriction may, for example, comprise limiting access such that only some actions may be performed by the identified operator and/or such that the identified operator can only use the analyzing device under supervision. Enforcement/release of the restriction may be exemplified correspondingly as the enforcement/release of the prohibition as described above. In some embodiments, a restriction may be enforced in response to releasing a prohibition; providing a trial period for the identified operator before granting full access to the analyzing device.

Controlling interaction with an operator device which is an analyzing device may comprise prohibiting performance of the analysis for further samples for the identifier of the operator. Thus, the identified operator may still have access to the analyzing device for performing (some) other types of analyses. Enforcement/release of the prohibition of analyses may be exemplified correspondingly as the enforcement/release of the prohibition of access to the analyzing device as described above.

Controlling interaction with an operator device which is an analyzing device may comprise increasing an amount of user interface rendered guidance instructions related to the analyzing device and/or relating to the analysis type for the identifier of the operator. Enforcement/release of the increased amount of guidance instructions may be exemplified correspondingly as the enforcement/release of the prohibition of access to the analyzing device as described above.

Controlling interaction with an operator device which is an analyzing device may comprise any suitable combination of the above examples.

Controlling interaction with an operator device which is an analyzing device may comprise enforcement/release as described above in relation to all analyzing devices 110, 140 reachable by the data handler 120. Alternatively or additionally, controlling interaction with an operator device which is an analyzing device may comprise enforcement/release as described above in relation to all analyzing devices 110, 140 (reachable by the data handler 120) which are of a same type, or which are of a same or more complicated type.

Controlling interaction with an operator device which is an analyzing device may comprise enforcement/release as described above in relation to all analyses. Alternatively or additionally, controlling interaction with an operator device which is an analyzing device may comprise enforcement/release as described above in relation to all analyses which are of a same type, or which are of a same or more complicated type.

Controlling interaction with an operator device which is a training device may comprise enforcing or prompting training (e.g., by sending a notification addressed to the identified operator) associated with the analyzing device and/or associated with the analysis, for the identifier of the operator. Enforcement/prompting of the training may be exemplified correspondingly as the enforcement of the prohibition of access to the analyzing device as described above. In some embodiments, training is enforced/prompted in combination with any of the above-described controlled interactions for analyzing devices. Then, release of the controlled interaction for analyzing devices may be responsive to detection that the training has been completed.

In some embodiments, the data handler also causes rendering of a notification based on the score value for one or more operator identifiers, and/or tracking of the score value over time for one or more operator identifiers. Rendering of the notification may be for the operator only, for a population of operators, and/or for an operator coordinator/supervisor.

Various approaches for the interaction control, which may be performed in isolation or in any suitable combination, are illustrated in FIG. 1 as using optional method steps and signaling.

In a first example approach, step 125 comprises sending a control instruction 197 to the training device 150, which is received by the training device 150 in step 155. In step 156, the interaction control is performed (e.g., enforcing/prompting/notifying/registering training for the identified operator). Alternatively or additionally, this approach may be applied for the first analyzing device 110 and/or for the second analyzing device 140.

In a second example approach, step 125 comprises sending a control instruction 196 to the second analyzing device 140, which is received by the second analyzing device 140 in step 145. In step 146, the interaction control is performed (e.g., enforcing prohibition, and/or restriction and/or increased amount of guidance instructions, for the identified operator). Alternatively or additionally, this approach may be applied for the first analyzing device 110.

In a third example approach, step 125 comprises receiving an indication 192 that the first analyzing device is triggered by an operator to perform an analysis of a sample, as illustrated by 111′ (compare with 111) and, in response thereto, sending a control instruction 195 to the first analyzing device 110, which is received by the first analyzing device 110 in step 115. In step 116, the interaction control is performed (e.g., enforcing prohibition, and/or restriction and/or increased amount of guidance instructions, for the identified operator). In association with the analysis of the sample, any sample associated handling errors are detected by the first analyzing device 110, as illustrated by 112′ (compare with 112). In response to detection of a handling error in step 112′, the first analyzing device 110 provides information regarding the detected handling error in association with an identifier of the operator, as illustrated by step 113′ (compare with 113). The information regarding the detected handling error associated with an identifier of the operator is illustrated as a signal 190′ (compare with 190). The information regarding any sample associated handling error detected by the triggered analyzing device is acquired by the data handler 120, as illustrated by step 123′ (compare with 123), and used to update handling error data associated with the identifier of the operator, as illustrated by step 124′ (compare with 124). Alternatively or additionally, this approach may be applied for the second analyzing device 140.

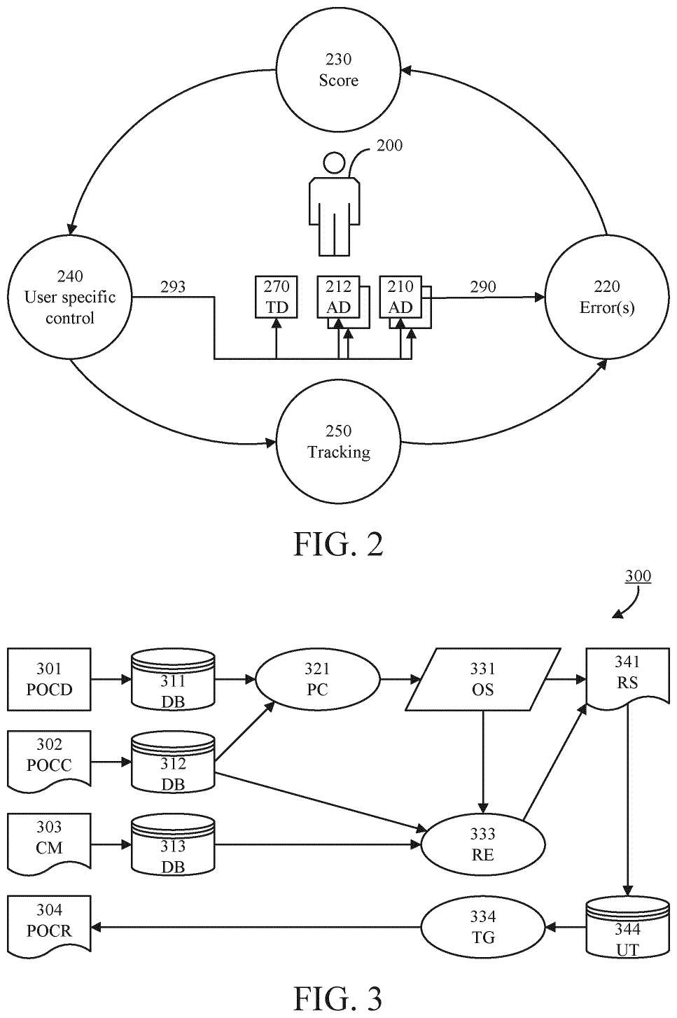

FIG. 2 schematically illustrates example mechanisms according to some embodiments. An operator 200 triggers (compare with 111, 111′ of FIG. 1) an analyzing device 210 (compare with 110 of FIG. 1) to perform an analysis of a biological sample and information 220 regarding any detected sample associated handling error (compare with 112, 112′ of FIG. 1) is reported (compare with 113, 123, 113′, 123′ of FIG. 1), as illustrated by 290. The information 220 is used to determine a score 230 (compare with 124, 124′ of FIG. 1). The score 230 is used to enforce user specific control 240 via control signaling 293 to a training device (TD) 270 and/or to a collection of analyzing devices (AD) comprising devices of the same type as the analyzing device 210 and/or other types of analyzing devices 212 (compare with 115, 125, 145, 155 of FIG. 1). Tracking 250 is performed of how the operator 200 responds to the user specific control 240 (e.g., whether training is successfully performed and/or whether the number of errors decreases) and may be used to adjust the handling error data 220.

FIG. 3 schematically illustrates example functional modules according to some embodiments. The point-of-care device (POCD) 301 exemplifies an analyzing device (compare with 110, 140 of FIG. 1), which causes handling error data to be stored in association with an operator identifier in a database (DB) 311 which exemplifies a storing device (compare with 130 of FIG. 1). The point-of-care coordinator (POCC) 302 embodies a supervisor role, which causes ranges/thresholds for handling error performance to be stored in a database (DB) 312. The performance calculator (PC) 321 embodies a mapping between handling error data and operator scores (OS) 331, based on the ranges/thresholds for handling error performance. The operator scores and the recommendation engine (RE) 333 provides rewards and/or suggestions (RS) 341, possibly based on the ranges/thresholds for handling error performance and/or on content provided by a content manager (CM) 303 and stored in a database (DB) 313. A user tracker (UT) 344 uses the rewards and/or suggestions 341 as input to a tracking generator (TG) 334, which provides key performance indicators (KPIs) in a point-of-care report (POCR) 304. One or more of the performance calculator 321, the recommendation engine 333, and the tracking generator 334 may be embodied within a data handler (compare with 120 of FIG. 1).

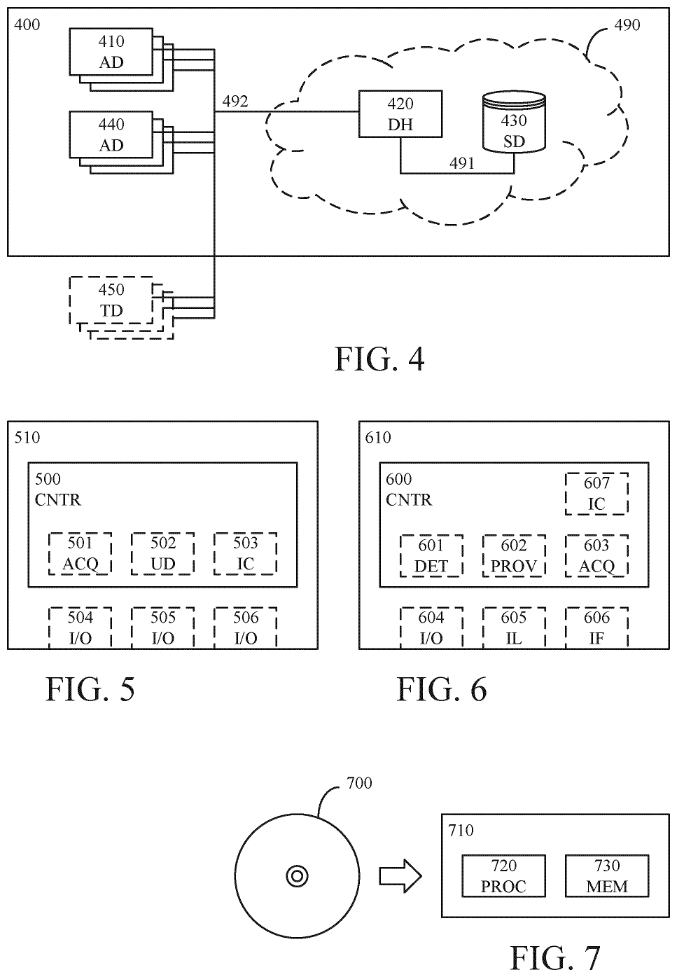

FIG. 4 schematically illustrates an example system 400 according to some embodiments. The example system 400 is for controlling operator interaction with one or more operator devices. One purpose of such control may be to control (e.g., reduce) the occurrence of sample handling errors in the context of using the analyzing devices. For example, one or more part of the system 400 may be configured to perform one or more method steps as described in connection with FIG. 1 (the details will not be repeated for FIG. 4).

The system comprises a data handler (DH) 420 (compare with 120 of FIG. 1), a storage device (SD) 430 (compare with 130 of FIG. 1), a first collection of analyzing devices (AD) 410 (compare with 110 of FIG. 1; e.g., analyzing devices of the same type), a second collection of analyzing devices (AD) 440 (compare with 140 of FIG. 1; e.g., analyzing devices of another type than 410). Optionally, the system 400 may also comprise, or be otherwise associated with (e.g., connected or connectable to) a collection of training devices (TD) 450 (compare with 150 of FIG. 1).

In some embodiments, the data handler 420 and/or the storage device 430 may be comprised in a cloud-based deployment, as illustrated by 490. For example, the data handler 420 and/or the storing device 430 may be comprised in a central device of a hospital information system (HIS) or a laboratory information system (LIS).

The data handler 420 and the storage device 430 may be comprised in the same device (e.g., a server), or may be comprised in different devices with an association of some sort (e.g., a wired or wireless connection). In any case the data handler 420 and the storage device 430 are configured to exchange information as indicated by 491 (compare with 191, 191′, 194 of FIG. 1).

The data handler 420 is also configured to exchange information with the analyzing devices 410, 440 and the training devices 450 via an association (e.g., a wired or wireless connection), as indicated by 492 (compare with 190, 190′, 192, 195, 196, 197 of FIG. 1).

FIG. 5 schematically illustrates an example apparatus 510 according to some embodiments. The apparatus 510 may, for example, be a data handler (e.g., any of the data handlers 120, 420 described in connection with FIGS. 1 and 4). Alternatively or additionally, the apparatus 510 be configured to perform, or cause performance of, one or more method steps as described in connection with FIG. 1 (the details will not be repeated for FIG. 5).

The apparatus 510 is for controlling operator interaction with one or more operator devices, wherein the one or more operator devices comprise one or more analyzing devices configured to analyze biological samples. The apparatus 510 comprises a controller (CNTR; e.g., controlling circuitry or a control module) 500. The apparatus 510 may also comprise one or more inputs/outputs (I/O; e.g., input/output circuitry or input/output module(s)) 504, 505, 506 configured for communication with analyzing devices and/or training devices and/or a storage device.

The controller 500 is configured to cause acquisition, in response to any of the one or more analyzing devices being triggered by the operator to perform an analysis of a sample, of information—in association with an identifier of the operator— regarding any sample associated handling error detected by the triggered analyzing device (compare with 123, 123′ of FIG. 1). The acquisition may, for example, be from an analyzing device. Alternatively or additionally, the acquisition may be performed via the input/output 504.

To this end, the controller 500 may comprise or be otherwise associated with (e.g., connected, or connectable, to) an acquirer (ACQ; e.g., acquiring circuitry or an acquisition module) 501. The acquirer 501 may be configured to acquire the information regarding detected sample associated handling error(s).

The controller 500 is also configured to cause dynamic update of handling error data associated with the identifier of the operator based on the information regarding detected handling errors (compare with 124, 124′ of FIG. 1). The update may, for example, be in a storage device. Alternatively or additionally, the update may be performed via the input/output 505.

To this end, the controller 500 may comprise or be otherwise associated with (e.g., connected, or connectable, to) an updater (UD; e.g., updating circuitry or an update module) 502. The updater 502 may be configured to dynamically update the handling error data.

The controller 500 is also configured to cause control, for the identifier of the operator, of interaction with at least one of the one or more operator devices based on the handling error data associated with the identifier of the operator (compare with 125 of FIG. 1). The control may, for example, comprise provision of control signaling to the operator device(s). Alternatively or additionally, the control may be performed via the input/output 506.

To this end, the controller 500 may comprise or be otherwise associated with (e.g., connected, or connectable, to) an interaction controller (IC; e.g., interaction controlling circuitry or an interaction control module) 503. The interaction controller 503 may be configured to control operator interaction with operator devices based on the handling error data.

FIG. 6 schematically illustrates an example operator device 610 according to some embodiments. The operator device 610 may, for example, be an analyzing device (e.g., any of the analyzing devices 110, 140, 410, 440 described in connection with FIGS. 1 and 4). Alternatively or additionally, the operator device 610 be configured to perform, or cause performance of, one or more method steps as described in connection with FIG. 1 (the details will not be repeated for FIG. 6).

The operator device 610 comprises a controller (CNTR; e.g., controlling circuitry or a control module) 600. The operator device 610 may also comprise one or more inputs/outputs (I/O; e.g., input/output circuitry or input/output module(s)) 604 configured for communication with a data handler. The operator device 610 may also comprise one or more inlets (IL) 605 configured for receiving a biological sample for analysis. The operator device 610 may also comprise one or more interfaces (IF) 606 configured for providing analysis result(s) and/or for operator interaction.

When the operator device is an analyzing device, the controller 600 is configured to cause detection, in response to the analyzing device being triggered by an operator to perform an analysis of a sample, of any sample associated handling error (compare with 112, 112′ of FIG. 1).

To this end, the controller 600 may comprise or be otherwise associated with (e.g., connected, or connectable, to) a detector (DET; e.g., detecting circuitry or a detection module) 601. The detector 601 may be configured to detect the handling error(s).

When the operator device is an analyzing device, the controller 600 is also configured to cause provision, in response to detection of a handling error, of information—in association with an identifier of the operator—regarding the detected handling error for dynamic updating of handling error data associated with the identifier of the operator (compare with 113, 113′ of FIG. 1). The provision may, for example, be to a data handler via the input/output 604.

To this end, the controller 600 may comprise or be otherwise associated with (e.g., connected, or connectable, to) a provisioner (PROV; e.g., provisioning circuitry or a provision module) 602. The provisioner 602 may be configured to provide the information regarding detected sample associated handling error(s).

When the operator device is a training device and/or an analyzing device, the controller 600 can be configured to cause acquisition of a control instruction associated with an identifier of an operator, wherein the control instruction is based on handling error data as updated based on information regarding previously detected handling errors associated with the identifier of the operator (compare with 115, 145, 155 of FIG. 1). The provision may, for example, be from a data handler via the input/output 604.

To this end, the controller 600 may comprise or be otherwise associated with (e.g., connected, or connectable, to) an acquirer (ACQ; e.g., acquiring circuitry or an acquisition module) 603. The acquirer 603 may be configured to acquire the control instruction.

When the operator device is a training device and/or an analyzing device, the controller 600 can also be configured to cause control of interaction with the operating device by the operator based on the control instruction (compare with 116, 146, 156 of FIG. 1). The operator interaction may, for example, be embodied via the interface 606.

To this end, the controller 600 may comprise or be otherwise associated with (e.g., connected, or connectable, to) an interaction controller (IC; e.g., interaction controlling circuitry or an interaction control module) 607. The interaction controller 607 may be configured to control operator interaction with the operator device based on the control instruction.

The described embodiments and their equivalents may be realized in software or hardware or a combination thereof. The embodiments may be performed by general purpose circuitry. Examples of general purpose circuitry include digital signal processors (DSP), central processing units (CPU), co-processor units, field programmable gate arrays (FPGA) and other programmable hardware. Alternatively or additionally, the embodiments may be performed by specialized circuitry, such as application specific integrated circuits (ASIC). The general purpose circuitry and/or the specialized circuitry may, for example, be associated with or comprised in an apparatus such as an operator device, an analyzing device, or a server.

Embodiments may appear within an electronic apparatus (such as an operator device, an analyzing device, or a server) comprising arrangements, circuitry, and/or logic according to any of the embodiments described herein. Alternatively or additionally, an electronic apparatus (such as an operator device, an analyzing device, or a server) may be configured to perform methods according to any of the embodiments described herein.

According to some embodiments, a computer program product comprises a tangible, or non-tangible, computer readable medium such as, for example a universal serial bus (USB) memory, a plug-in card, an embedded drive or a read only memory (ROM). FIG. 7 illustrates an example computer readable medium in the form of a compact disc (CD) ROM 700. The computer readable medium has stored thereon a computer program comprising program instructions. The computer program is loadable into a data processor (PROC; e.g., data processing circuitry or a data processing unit) 720, which may, for example, be comprised in a an operator device, an analyzing device, or a server 710. When loaded into the data processor, the computer program may be stored in a memory (MEM) 730 associated with or comprised in the data processor. According to some embodiments, the computer program may, when loaded into and run by the data processor, cause execution of method steps according to, for example, any of the methods illustrated in FIG. 1, or otherwise described herein.

Generally, all terms used herein are to be interpreted according to their ordinary meaning in the relevant technical field, unless a different meaning is clearly given and/or is implied from the context in which it is used.

Reference has been made herein to various embodiments. However, a person skilled in the art would recognize numerous variations to the described embodiments that would still fall within the scope of the claims.

For example, the method embodiments described herein discloses example methods through steps being performed in a certain order. However, it is recognized that these sequences of events may take place in another order without departing from the scope of the claims. Furthermore, some method steps may be performed in parallel even though they have been described as being performed in sequence. Thus, the steps of any methods disclosed herein do not have to be performed in the exact order disclosed, unless a step is explicitly described as following or preceding another step and/or where it is implicit that a step must follow or precede another step.

In the same manner, it should be noted that in the description of embodiments, the partition of functional blocks into particular units is by no means intended as limiting. Contrarily, these partitions are merely examples. Functional blocks described herein as one unit may be split into two or more units. Furthermore, functional blocks described herein as being implemented as two or more units may be merged into fewer (e.g. a single) unit.

Any feature of any of the embodiments disclosed herein may be applied to any other embodiment, wherever suitable. Likewise, any advantage of any of the embodiments may apply to any other embodiments, and vice versa.

Hence, it should be understood that the details of the described embodiments are merely examples brought forward for illustrative purposes, and that all variations that fall within the scope of the claims are intended to be embraced therein.

1. A computer-implemented method of controlling operator interaction with one or more operator devices, wherein the one or more operator devices comprise one or more analyzing devices configured to analyze biological samples, the method comprising:
responsive to any of the one or more analyzing devices being triggered by the operator to perform an analysis of a sample,
acquiring, in association with an identifier of the operator, information regarding any sample associated handling error detected by the triggered analyzing device;
dynamically updating handling error data associated with the identifier of the operator based on the information regarding detected handling errors; and
controlling, for the identifier of the operator, interaction with at least one of the one or more operator devices based on the handling error data associated with the identifier of the operator, wherein controlling interaction with at least one of the one or more operator devices comprises one or more of:
prohibiting or restricting further access to the one or more analyzing devices, for the identifier of the operator;
prohibiting performance of the analysis for further samples, for the identifier of the operator;
increasing an amount of user interface rendered guidance instructions related to the one or more analyzing devices and/or relating to the analysis, for the identifier of the operator; and
enforcing or prompting training associated with the analyzing device and/or associated with the analysis, for the identifier of the operator.

2. The method of claim 1, wherein the information regarding a handling error comprises indication of one or more of: detection of the handling error, and an error type of the handling error.

3. The method of claim 1, further comprising acquiring, in association with the identifier of the operator and the information regarding a handling error, one or more of:
identification of the analyzing device triggered by the operator to perform the analysis,
identification of an equipment type of the analyzing device triggered by the operator to perform the analysis, and
identification of an analysis type of the analysis performed.

4. The method of claim 3, wherein dynamically updating the handling error data associated with the identifier of the operator is further based on one or more of: the identification of the analyzing device triggered by the operator to perform the analysis, the identification of the equipment type of the analyzing device triggered by the operator to perform the analysis, and the identification of the analysis type of the analysis performed.

5. The method of claim 1, wherein controlling interaction with at least one of the one or more operator devices comprises:
determining a score value for the identifier of the operator based on the dynamically updated handling error data; and
controlling interaction with at least one of the one or more operator devices based on the score value.

6. The method of claim 5, further comprising one or more of:
rendering a notification based on the score value for one or more operator identifiers; and
tracking the score value over time for one or more operator identifiers.

7. A computer-implemented method of an analyzing device configured to analyze biological samples, for controlling operator interaction with one or more operator devices, wherein the one or more operator devices comprise the analyzing device, the method comprising:
responsive to the analyzing device being triggered by the operator to perform an analysis of a sample, detecting any sample associated handling error; and
in response to detection of a handling error, providing, in association with an identifier of the operator, information regarding the detected handling error for dynamic updating of handling error data associated with the identifier of the operator, wherein the handling error data associated with the identifier of the operator is for controlling interaction with at least one of the one or more operator devices, for the identifier of the operator, wherein controlling interaction with at least one of the one or more operator devices comprises one or more of:
prohibiting or restricting further access to the one or more analyzing devices, for the identifier of the operator;
prohibiting performance of the analysis for further samples, for the identifier of the operator;
increasing an amount of user interface rendered guidance instructions related to the one or more analyzing devices and/or relating to the analysis, for the identifier of the operator; and
enforcing or prompting training associated with the analyzing device and/or associated with the analysis, for the identifier of the operator.

8. The method of claim 7, further comprising, responsive to the analyzing device being triggered by the operator to perform the analysis of the sample:
acquiring a control instruction associated with the identifier of the operator, wherein the control instruction is based on handling error data as updated based on information regarding previously detected handling errors associated with the identifier of the operator; and
controlling interaction with the analyzing device by the operator based on the control instruction.

9. A computer program product comprising a non-transitory computer readable medium, having thereon a computer program comprising program instructions, the computer program being loadable into a data processing unit and configured to cause execution of the method according to claim 1 when the computer program is run by the data processing unit.

10. An apparatus for controlling operator interaction with one or more operator devices, wherein the one or more operator devices comprise one or more analyzing devices configured to analyze biological samples, the apparatus comprising controlling circuitry configured to cause:
acquisition, in response to any of the one or more analyzing devices being triggered by the operator to perform an analysis of a sample, of information, in association with an identifier of the operator, regarding any sample associated handling error detected by the triggered analyzing device;
dynamic update of handling error data associated with the identifier of the operator based on the information regarding detected handling errors; and
control, for the identifier of the operator, of interaction with at least one of the one or more operator devices based on the handling error data associated with the identifier of the operator, wherein control of interaction with at least one of the one or more operator devices comprises one or more of:
prohibition or restriction of further access to the one or more analyzing devices, for the identifier of the operator;
prohibition of performance of the analysis for further samples, for the identifier of the operator;
increase of an amount of user interface rendered guidance instructions related to the one or more analyzing devices and/or relating to the analysis, for the identifier of the operator; and
enforcement or prompt of training associated with the analyzing device and/or associated with the analysis, for the identifier of the operator.

11. A server comprising the apparatus of claim 10.

12. A storage device carrying handling error data for controlling operator interaction with one or more operator devices according to claim 1, wherein the one or more operator devices comprise one or more analyzing devices configured to analyze biological samples, wherein the handling error data is associated with respective identifiers of a plurality of operators, and is based on information regarding sample associated handling errors detected responsive to any of the one or more analyzing devices being triggered by an operator to perform an analysis of a sample.

13. An analyzing device configured to analyze biological samples, the analyzing device comprising controlling circuitry configured to cause:
detection, in response to the analyzing device being triggered by an operator to perform an analysis of a sample, of any sample associated handling error; and provision, in response to detection of a handling error, of information, in association with an identifier of the operator, regarding the detected handling error for dynamic updating of handling error data associated with the identifier of the operator, wherein the handling error data associated with the identifier of the operator is for controlling interaction with at least one of one or more operator devices, for the identifier of the operator, wherein the one or more operator devices comprise the analyzing device, wherein controlling interaction with at least one of the one or more operator devices comprises one or more of:
prohibiting or restricting further access to the one or more analyzing devices, for the identifier of the operator;
prohibiting performance of the analysis for further samples, for the identifier of the operator;
increasing an amount of user interface rendered guidance instructions related to the one or more analyzing devices and/or relating to the analysis, for the identifier of the operator; and
enforcing or prompting training associated with the analyzing device and/or associated with the analysis, for the identifier of the operator.

14. An operator device, wherein the operator device is an analyzing device configured to analyze biological samples and/or a training device configured to enable sample handling training for biological sample analysis, the operator device comprising controlling circuitry configured to cause:
acquisition of a control instruction associated with an identifier of an operator, wherein the control instruction is based on handling error data as updated based on information regarding previously detected handling errors associated with the identifier of the operator; and
control of interaction with the operating device by the operator based on the control instruction, wherein control of interaction with at least one of the one or more operator devices comprises one or more of:
prohibition or restriction of further access to the one or more analyzing devices, for the identifier of the operator;
prohibition of performance of the analysis for further samples, for the identifier of the operator;
increase of an amount of user interface rendered guidance instructions related to the one or more analyzing devices and/or relating to the analysis, for the identifier of the operator; and
enforcement or prompt of training associated with the analyzing device and/or associated with the analysis, for the identifier of the operator.

15. A system for controlling operator interaction with one or more operator devices, wherein the one or more operator devices comprise one or more analyzing devices configured to analyze biological samples, the system comprising:
a server comprising an apparatus for controlling operator interaction with one or more operator devices, wherein the one or more operator devices comprise one or more analyzing devices configured to analyze biological samples, the apparatus comprising controlling circuitry configured to cause:
acquisition, in response to any of the one or more analyzing devices being triggered by the operator to perform an analysis of a sample, of information, in association with an identifier of the operator, regarding any sample associated handling error detected by the triggered analyzing device;
dynamic update of handling error data associated with the identifier of the operator based on the information regarding detected handling errors; and
control, for the identifier of the operator, of interaction with at least one of the one or more operator devices based on the handling error data associated with the identifier of the operator, wherein control of interaction with at least one of the one or more operator devices comprises one or more of:
prohibition or restriction of further access to the one or more analyzing devices, for the identifier of the operator;
prohibition of performance of the analysis for further samples, for the identifier of the operator;
increase of an amount of user interface rendered guidance instructions related to the one or more analyzing devices and/or relating to the analysis, for the identifier of the operator; and
enforcement or prompt of training associated with the analyzing device and/or associated with the analysis, for the identifier of the operator;
a storage device carrying handling error data for controlling operator interaction with one or more operator devices according to claim 1, wherein the one or more operator devices comprise one or more analyzing devices configured to analyze biological samples, wherein the handling error data is associated with respective identifiers of a plurality of operators, and is based on information regarding sample associated handling errors detected responsive to any of the one or more analyzing devices being triggered by an operator to perform an analysis of a sample; and
at least one analyzing device configured to analyze biological samples, the analyzing device comprising controlling circuitry configured to cause:
detection, in response to the analyzing device being triggered by an operator to perform an analysis of a sample, of any sample associated handling error; and
provision, in response to detection of a handling error, of information, in association with an identifier of the operator, regarding the detected handling error for dynamic updating of handling error data associated with the identifier of the operator, wherein the handling error data associated with the identifier of the operator is for controlling interaction with at least one of one or more operator devices, for the identifier of the operator, wherein the one or more operator devices comprise the analyzing device, wherein controlling interaction with at least one of the one or more operator devices comprises one or more of:
prohibiting or restricting further access to the one or more analyzing devices, for the identifier of the operator;
prohibiting performance of the analysis for further samples, for the identifier of the operator;
increasing an amount of user interface rendered guidance instructions related to the one or more analyzing devices and/or relating to the analysis, for the identifier of the operator; and
enforcing or prompting training associated with the analyzing device and/or associated with the analysis, for the identifier of the operator.
